(19) United States
(12) Patent Application Publication
(10) Pub. No.: US 2016/0330355 A1
(43) Pub. Date: Nov. 10, 2016

(54) Intra-oral scanner with color tip assembly

(71) Applicant: D4D Technologies, LLC, Richardson, TX (US)

(72) Inventors: Andrei Tchouprakov, Plano, TX (US); Greg Basile, Dallas, TX (US); Mark S. Quadling, Plano, TX (US); Henley S. Quadling, Dallas, TX (US); Rod Duncan, Dallas, TX (US); Ye Li, Plano, TX (US); Grant Kenworthy, Dallas, TX (US)

(21) Appl. No.: 15/065,542

(22) Filed: Mar. 9, 2016

Related U.S. Application Data
(60) Provisional application No. 62/130,320, filed on Mar. 9, 2015.

(51) Int. Cl. H04N 5/225; A61C 9/00; A61B 1/24; H04N 5/372

(52) U.S. Cl. CPC H04N 5/2256; H04N 5/372; A61C 9/0053; A61B 1/24; H04N 2005/2255

(57) ABSTRACT

A technique to enable an existing monochrome camera in an intra-oral scanner to capture color images without making hardware changes to the camera. This operation is achieved by retrofitting a “tip” assembly of the scanner with red, green and blue light emitting diodes (LEDs), and then driving those diodes to illuminate the scene being captured by the scanner. Electronics in or associated with the scanner are operative to synchronize the LEDs to the frame capture of the monochrome camera in the device. A color image is created by combining the red-, green- and blue-illuminated images. Thus, color imagery is created from a monochrome camera and, in particular, by illuminating the screen with specific colors while the camera captures images. In this manner, single colored images are captured and combined into full color images. The system captures the color images with full resolution and sensitivity, thus producing higher quality full color images.

### BACKGROUND OF THE INVENTION

1. Technical Field

This disclosure relates generally to computer-assisted techniques for creating dental restorations.

2. Brief Description of the Related Art

During the last decade various technological advancements have increasingly started to be applied to systems in the healthcare arena, particularly in dental care. More specifically for example, traditional imaging and computer vision algorithms coupled with soft X-ray sensitive charge coupled device (CCD) based vision hardware have rendered conventional X ray photography ubiquitous, while more advanced data imaging and processing has enabled passive intraoral 3D topography. The latter comprises the acquisition portion of a CAD/CAM system, which would typically be followed by a design step using some sort of manipulating software, and a manufacturing step that might entail an office laser printer-sized milling machine. The entire system allows a dentist to provide a patient the same services a manufacturing laboratory would provide with a certain turnaround time, however, all chair-side and on-the-spot, greatly reducing the possibility of infections and discomfort to the patient. In addition, clinical cases containing raw and processed data are easily shared as digital files between dentists who lack the second portion of the system, i.e. the manufacturing step, and laboratories who have adapted and evolved to embrace CAD/CAM.

The CAD/CAM system described typically includes an intra-oral scanner that uses a monochrome 3D camera. Although these systems provide significant advantages, it has not been possible to capture color images using such devices without making hardware changes to the camera. Traditional color cameras create colored images by applying color filters in front of the camera's sensing pixels. A conventional approach of this type may be used in an intra-oral scanner, but the solution is complex and costly to implement. In addition, it lowers the signal-to-noise ratio and the color resolution of the camera.

### BRIEF SUMMARY

This disclosure describes a technique to enable an existing monochrome camera in an intra-oral scanner to capture color images without making hardware changes to the camera. Preferably, this operation is achieved by retrofitting a “tip” assembly of the scanner with red, green and blue light emitting diodes (LEDs), and then driving those diodes to illuminate the scene being captured by the scanner. Electronics in or associated with the scanner are operative to synchronize the LEDs to the frame capture of the monochrome camera in the device. A color image is then created by combining the red-, green- and blue-illuminated images. Thus, according to this disclosure color imagery is created from a monochrome camera and, in particular, by illuminating the screen with specific colors while the camera captures images. The color of the illumination is changed as needed. In this manner, single colored images are captured and combined into full color images. The monochrome camera and color tip assembly (and associated electronics) captures the color images with full resolution and sensitivity, thus producing higher quality full color images.

The foregoing has outlined some of the more pertinent features of the subject matter. These features should be construed to be merely illustrative.

### BRIEF DESCRIPTION OF THE DRAWINGS

For a more complete understanding of the disclosed subject matter and the advantages thereof, reference is now made to the following descriptions taken in conjunction with the accompanying drawings, in which:

FIG. 1 illustrates basic components and geometry underlying 3D triangulation;

FIG. 2 is a known technique to project laser pattern lines onto a preparation area using an intra-oral hand-held wand device;

FIG. 3 illustrates a 3D generated model created by processing the partially-illuminated pattern lines;

FIG. 4 illustrates an optical sub-system of an intra-oral scanning device of this disclosure with its outer housing removed;

FIG. 5 is an elevation view of the intra-oral scanning device of this disclosure illustrating a removable tip that includes a heating element;

FIG. 6 is an embodiment of system architecture to control the hand-held intra-oral device of this disclosure;

FIG. 7 illustrates a preferred 3D pipeline processing approach implemented in the device;

FIG. 8 illustrates the rendering of a textured 3D model juxtaposed against a live video feed provided by the scanning techniques of this disclosure;

FIG. 9 is an elevation view of the scanning device; and

FIG. 10 depicts an alternative embodiment of the intra-oral scanning device wherein the detachable tip of the assembly is modified to house red, green and blue light emitting diodes (LEDs) that illuminate the scene being captured by the device.

### DETAILED DESCRIPTION

As described above, this disclosure provides a way in which an existing monochrome camera, e.g., in an intra-oral scanner, can be used to capture color images without making hardware changes to the camera itself. As will be seen, in a preferred implementation this advantage is achieved by retrofitting a “tip” assembly of the intra-oral scanner with red, green and blue light emitting diodes (LEDs), and then driving those diodes to illuminate the scene being captured by the scanner. Electronics in or associated with the scanner are then operative to synchronize the LEDs to the frame capture of the monochrome camera in the device. A color image is then created by combining the red-, green- and blue-illuminated images. Thus, according to this disclosure color imagery is created from a monochrome camera and, in particular, by illuminating the screen with specific colors while the camera captures images. In this manner, single colored images are captured and combined into full color images. The camera captures the color images with full resolution and sensitivity, thus producing higher quality full color images.

### Intra-Oral Scanning System and Method

By way of background, the following section describes a known commercial intra-oral scanning system and method in which the technique of this disclosure may be implemented.

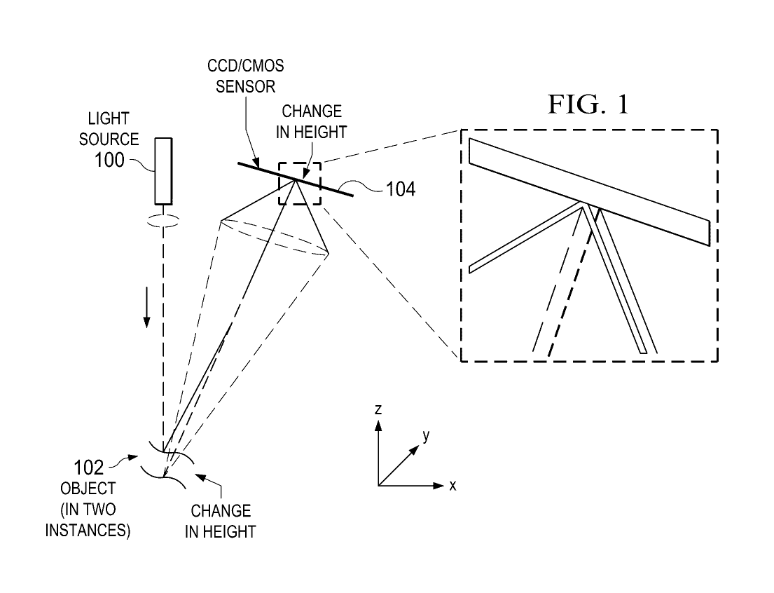

The principles behind structured light based 3D triangulation are explained in various works. The underlying principles are described with respect to FIG. 1, which illustrates a light source 100 directed to an object 102, with the reflection being captured a charge coupled device (CCD) imaging surface 104. This illustrates the basic components and principles behind 3D triangulation in an intuitive manner. In this approach, a change in height due to object topography is registered as a deviation of a projected point onto a charge coupled device (CCD) imaging surface. In operation, a laser pattern is projected with the help of an LCOS (i.e. liquid crystal on silicon) device. In particular, a sequence of a set of lines is generated by the lines reflected from LCOS to form a set of planes, or, if distortion is involved (as typically is the case when implemented), a set of conical or ruled surfaces.

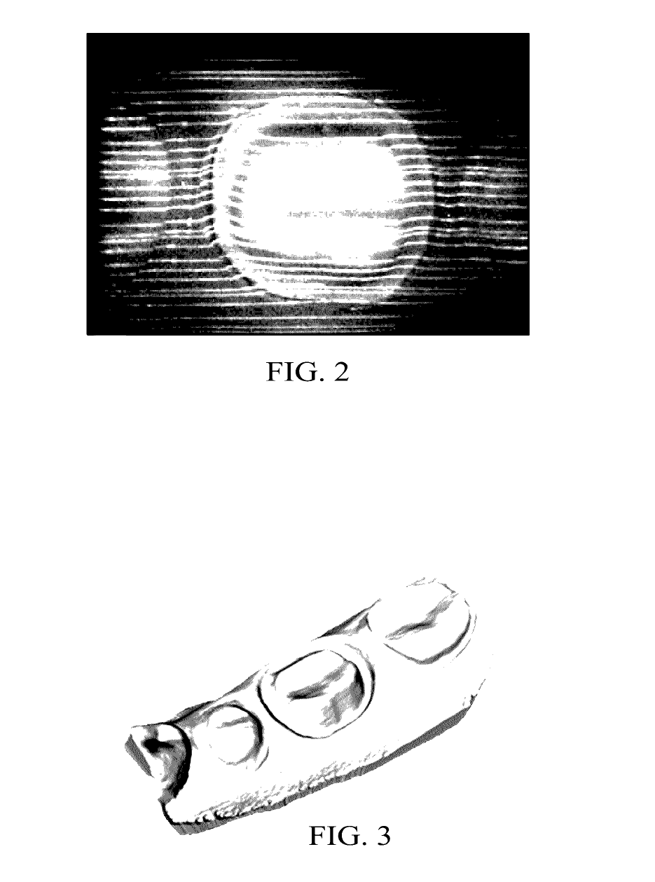

FIG. 2 illustrates a pattern projected onto a preparation area. In an analogous manner, each point in the camera CCD frame corresponds to a line in space that passes through the imaging center or focal point. Because preferably the LCOS and the camera are laterally separated, the point of intersection between each laser surface generated by a single LCOS pixel and each line of sight is well-defined. Thus, by knowing the pixel coordinates on the camera matrix and the shape of the laser surface, it is possible to obtain coordinates of a 3D point corresponding to that pixel. When laser lines are projected onto the surface of the scanned object, the image of those lines in the camera plane defines a set of 3D points corresponding to the object surface. To obtain the shape of the surfaces formed to each laser line, a calibration procedure is performed. A camera lens calibration is performed by taking an image of a checkerboard pattern, with a set of intrinsic camera parameters (such as focal length and lens distortion) estimated as a result. From this, an exact direction of a ray corresponding to each camera pixel is established. To determine the shape of the laser surfaces, a set of planes located at the known distances with known orientation are scanned. Each line projected onto each successive plane forms an image on the CCD matrix, represented as a set of pixels and, because for each pixel the corresponding direction and the actual distance to the calibration plane are known, the set of 3D coordinates forming a line of intersection between a laser surface and calibration plane are known as well. Interpolation between successive lines produces the shape of the laser surface, represented by the final generated 3D model shown in FIG. 3.

The frames used to capture the data for the 3D model are partially-illuminated frames (such as shown in FIG. 2, wherein the LCOS paints a series of lines in a pattern). According to this disclosure, and to facilitate the operation of the device and provide live video as feedback to the operator (as well as the 3D-computed data), a preferred implementation uses a sequence of patterns throughout which full illumination frames are selectively interspersed. A full illumination frame involves all or substantially all lines being turned on, as compared to the partially-illuminated approach shown in FIG. 2, wherein only some lines are projected. In a full illumination frame, in effect there is no pattern. The partially-illustrated frames provide the data from which the 3D coordinates of the surface are determined. A technique for rendering frames in this manner is described in U.S. Pat. No. 7,184,150, the disclosure of which is incorporated herein by reference. In contrast, the full illumination frames are used for texturing the 3D model generated by the partially-illuminated frame data. In one sequence, a first set (e.g., six) pattern frames are used, interspersed with a second set (e.g., three) illumination frames, for a sequence total of nine total CCD frames. A software traffic shaper is then used to separate captured frames in two streams, namely, a live preview stream, and a data processing stream from which the 3D model is generated. If necessary, e.g., for computational or storage efficiencies, the live preview stream can give up priority and drop some frames when the CPU work load exceeds a certain limit.

In the embodiment described above, the same light source (e.g., a blue laser) is used to generate both the first series of frames and the second series of (interleaved) frames, and a monochrome sensor is used. If it is desired to output a color video preview, one or more other light sources (e.g., a red laser, a green laser, or some combination) are used to vary the color of the full illumination frames. Thus, in one alternative embodiment, there are three different light sources (blue, red and green), with the resulting data returned from these full illumination frames then being used to provide a color video preview. As yet another alternative, full illumination frames are generated using a source of monochrome light, and a color sensor is used to receive the reflected data (to generate the color video preview). Still another alternative to generate a color video image is to use full illumination red and green frames with a partial illumination blue frame. Other light sources (e.g., a red/green laser or even an LED) may obviate the full illumination blue frame. Another possibility is to use red as the additional color (leaving out the green, or vice versa), and then processing the resulting data to generate a pseudo-color video stream. When the approach uses the red, green and blue laser, the scanner may be used to generate a simplified optical coherence tomography (OCT) scan using discrete lasers instead of a single broadband source, or a swept source.

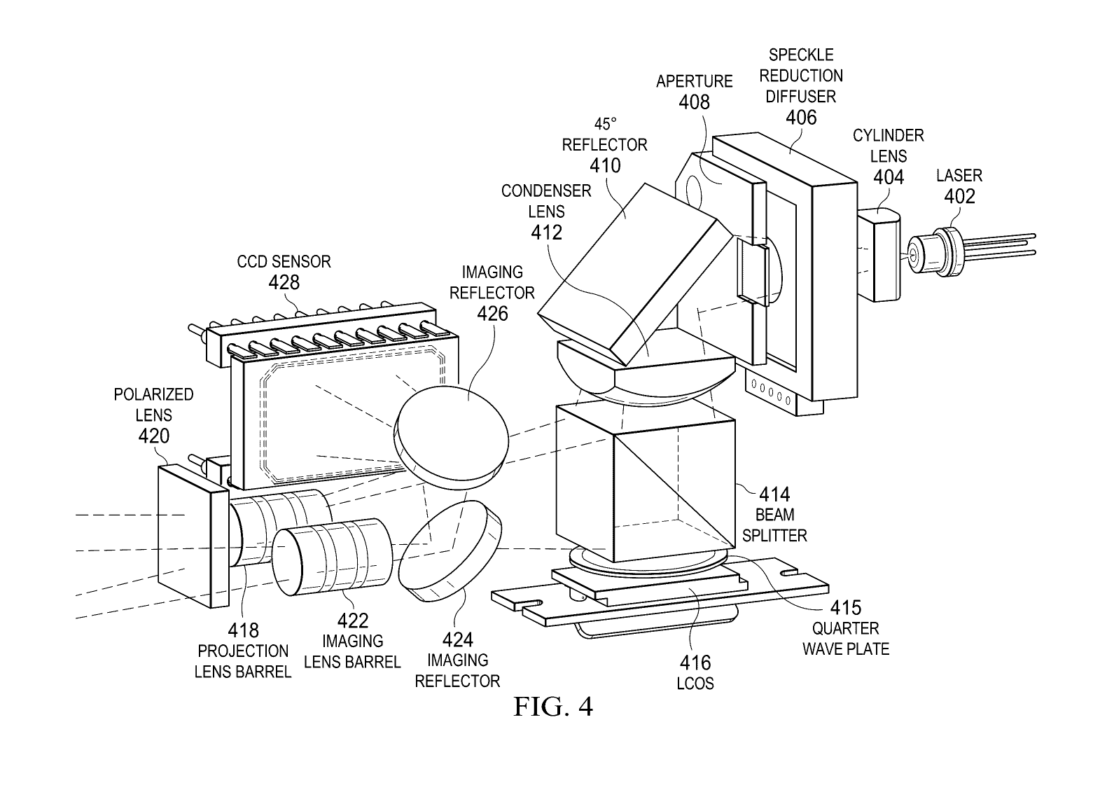

FIG. 4 illustrates an embodiment of an optical sub-system of an intra-oral device with its outer housing removed. The primary imaging components of the optical sub-system 400 include a laser 402, a cylinder lens 404, a speckle reduction diffuser 406, an aperture 408, a reflector 410, a condenser lens 412, a beam splitter 414, a quarter wave plate 415, the LCOS device assembly 416, a projection lens barrel assembly 418, and a polarized lens 420. A return (imaging) path comprises imaging lens barrel assembly 422, first and second imaging reflectors 424 and 426, and the CCD sensor 428.

Without meant to be limiting, a preferred laser is a blue laser device with a wavelength of 450 nm, and thus the optical path for the projection side is polarization-based. In this embodiment, projection is achieved with the LCOS device 416 having a resolution of 800 by 600 pixels and a pixel size of 8.0 um. The speckle reduction diffuser (a de-speckle component) is used to eliminate the speckle issues otherwise caused by using a laser as the light source. Using a laser (instead of, for example, an LED light source) produces a much brighter projected pattern which, in turn, allows the scanner to image intra-orally without powder.

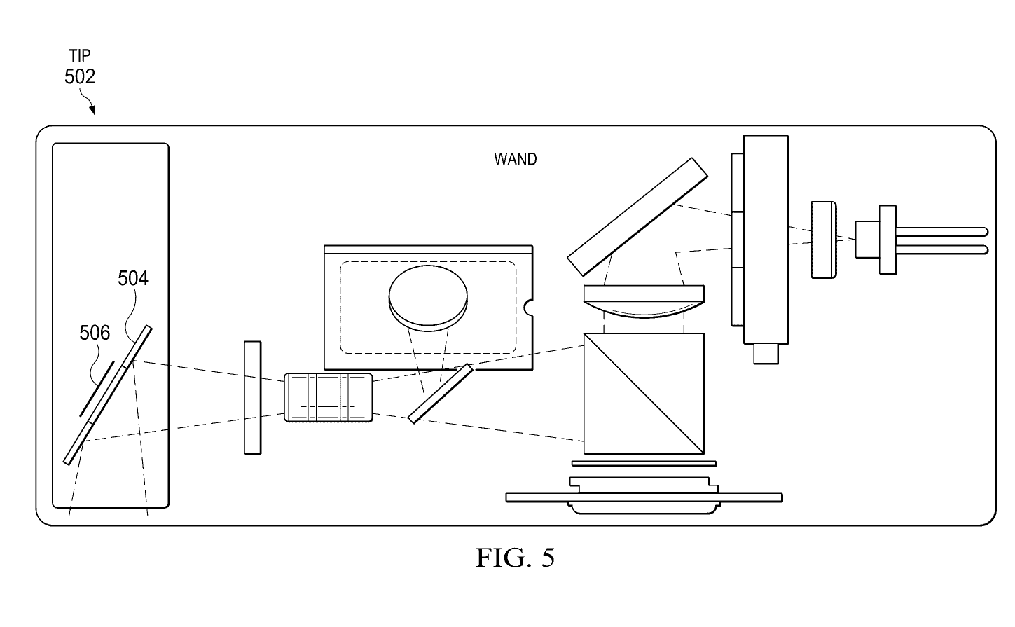

As seen in FIG. 5, the intra-oral device 500 is configured as a hand-held wand that includes a tip portion or “tip” 502. FIG. 9 illustrates an embodiment of the wand with the outer housing present. As seen in FIG. 5, the tip 502 includes a mirror 504 and preferably no additional glass windows; the mirror 504 reflects the projection path from a long axis of the device (the optical sub-system shown in FIG. 4) towards the target area being scanned, and that receives the imaging path data returned from the target area. The returned data is forwarded down the long axis of the device, where it is imaged by the CCD sensor device. By using a mirror 504 in the tip 502, the possibility of a surface near the target area being contaminated with dirt or fluid is reduced. This is desirable, as any contamination on a glass window or prism surface may be close to (or within) a focused region of the optical path, and therefore may result in erroneous measurements. The reflecting mirror 504 is outside the focus region, and thus any slight imperfections or debris on its surface will not result in erroneous data measurements. Preferably, the tip 502 is removable from the rest of the wand housing, and the mirror is heated (with an active heating element 506) to prevent fogging of the optical surfaces while the device is being deployed intra-orally. The heating element may be a metal conductive element that is supported in a molded plastic housing and that receives current from other wand electronics. Any other type of heating element may be used. FIG. 9 illustrates the removable tip 902. In this manner, multiple tips (the others now shown), each with varying mirror angles and sizes, may be implemented with a single wand body that includes the optical sub-system shown in FIG. 4. In this manner, different tips may be used for different scanning scenarios, such as scanning posterior preparations in small patients, or more challenging situations where a steeper viewing angle is required.

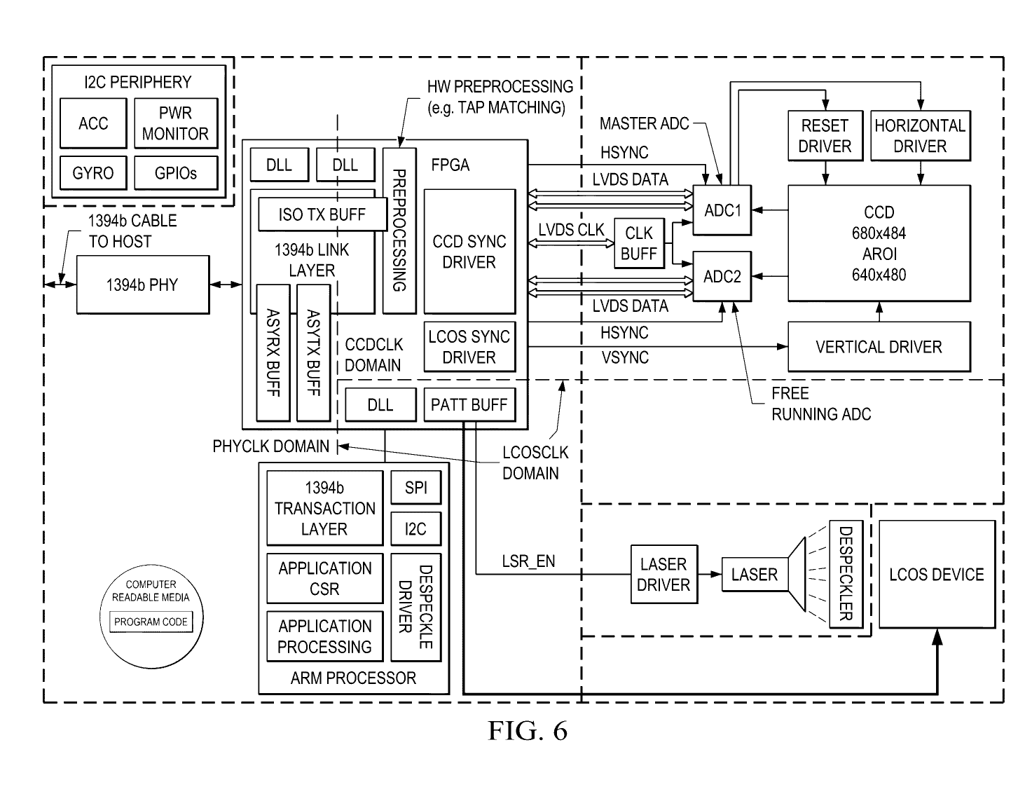

FIG. 6 illustrates system architecture for the wand. In this implementation there are three (3) subsystems, namely, an imaging sub-system, a projection/illumination sub-system, and a periphery sub-system. Preferably, imaging is achieved by an over-clocked dual-tap CCD with an active resolution of 648 by 484 pixels, and a pixel size of 9 um.

In this embodiment, which is not intended to be limiting, the system architecture comprises a tightly-integrated IP FPGA core containing an IEEE 1394b 5800 link layer, CCD/ADC synchronizers, the LOCS and illumination synchronizer. Cross-clock domain FIFOs are implemented to synchronize the CCD exposure/LCOS projection/CCD readout sequence to the IEEE1394 bus clock, which is 125 us or 8000 Hz. The FPGA is assisted by an ARM processor, implementing the IEEE1394b transaction layer and various housekeeping system tasks, such as running an I2C periphery priority task scheduler. The FPGA implements deep FIFOs for asynchronous packet reception and transmission and likewise for the CCD video data, which is sent as isochronous packets. It also implements a prioritized interrupt mechanism that enables the ARM processor to de-queue and en-queue IEEE1394 asynchronous packets and to complete them according to the bus transaction layer specification and various application requirements. The bulk of the housekeeping work in the system originates in user space software, ends up as an asynchronous packet in the ARM processor and is dispatched from there through either I2C or SPI to the appropriate peripheral component. The software is designed to maintain the hardware pipelining while running within a non-real time operating system (OS), such as Microsoft® Windows 7 and Apple® OS/X. Other operating systems such as Android or iOS® may be used.

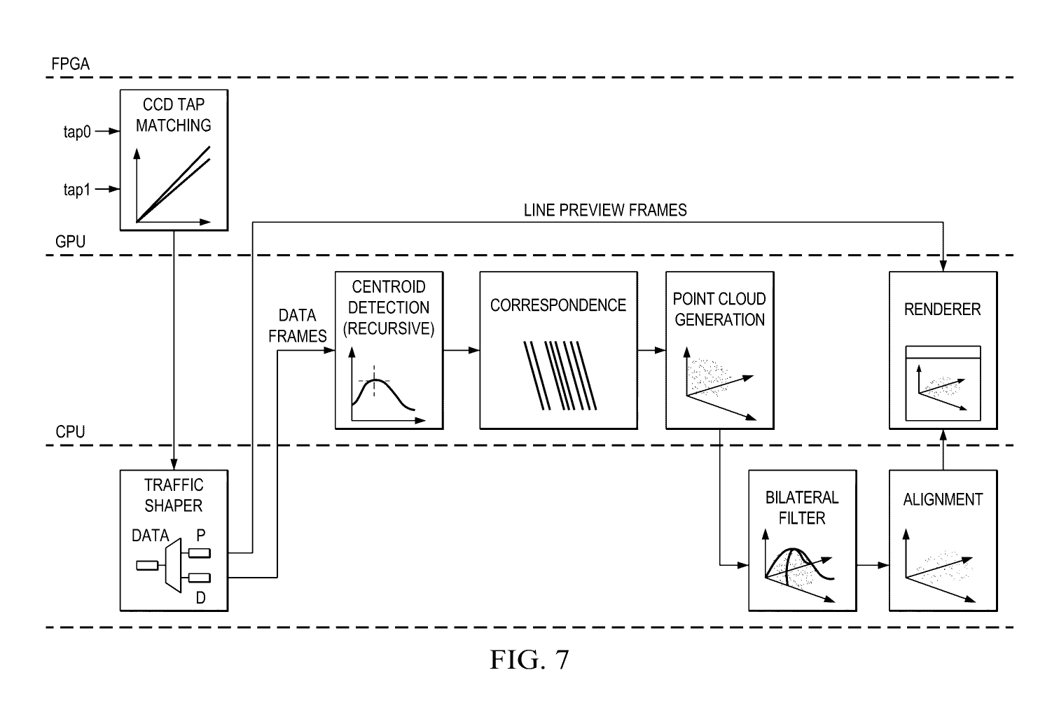

In this embodiment, and to provide the required data quality at a desired rate, the imaging system preferably is comprised of a slightly over-clocked dual tapped CCD. The CCD is 680 by 484 pixels containing some dark columns and rows for black offset correction and is specified to have 57 dB of dynamic range at a pixel clock of 20 MHz with a maximum pixel clock of 30 MHz. The projection and illumination subsystem comprises LCOS device, a laser diode driver, a 450 nm blue laser diode and an optical de-speckling device. As illustrated in FIG. 7, preferably data is processed in a pipeline distributed across several computing resources. In this approach, data from the CCD ADCs, 8 bit per pixel, is first run through a tap matching block where both taps are linearized and matched according to a look up table. This implies a previous calibration step. The traffic shaper separates the data into live preview and 3D processing input frames. The 3D processing input frames contain projected patterns. On the GPU these frames are first run through a centroid detector implemented as a recursive sub-pixel edge detector, a correspondence block, and finally a point cloud generation block. This output is then run on the CPU side through a bilateral filter for data smoothing, and through an alignment block to stitch scans together. This processing distribution allows for running alignment in a pipelined fashion with 3D point cloud generation happening in parallel.

Preferably, fast imaging is used to allow minimization of errors (e.g., due to operator hand jitter). In one embodiment, good results were obtained with a live preview window of approximately 20 frames per second, coupled with approximately 15 frames per second for the 3D data.

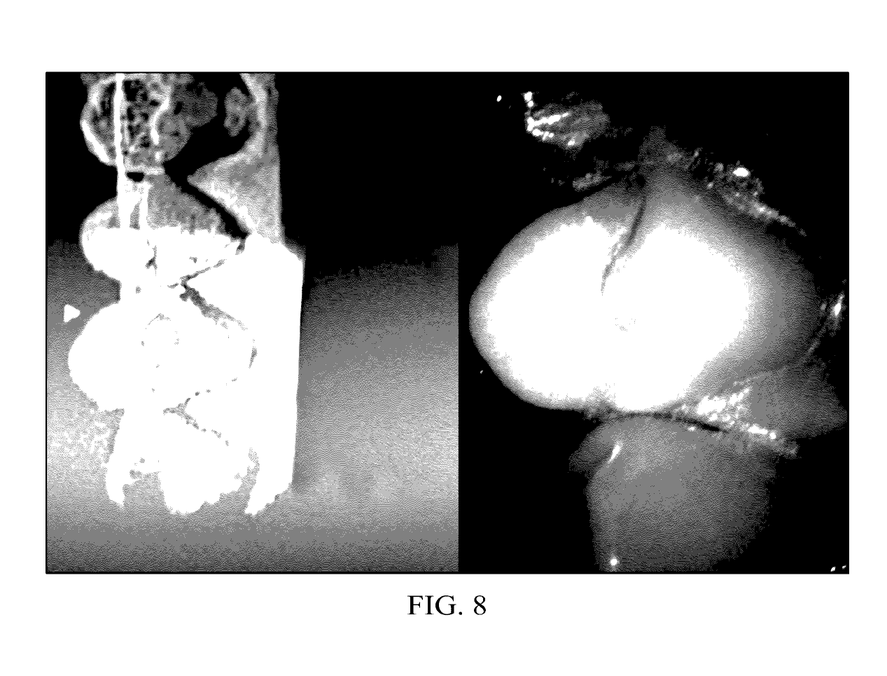

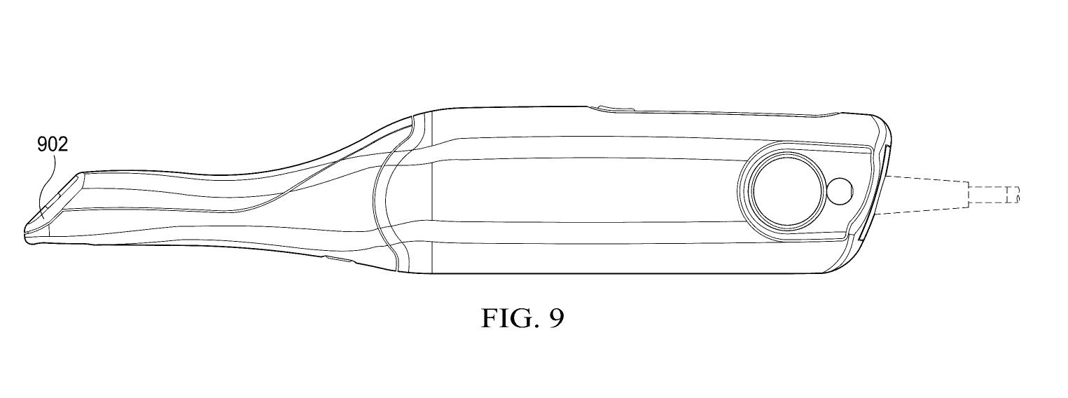

A representative display interface is used to display the 3D model, on the one hand, and the live video preview window, on the other. FIG. 8 illustrates a representative screen grab from a juxtaposition of these views. These views may be juxtaposed in any convenient display format (e.g., side-by-side, above-below, as an overlay (or “3D texture” view), or the like).

### Providing Full Color Images Using a Monochrome Camera and a LED Tip Assembly

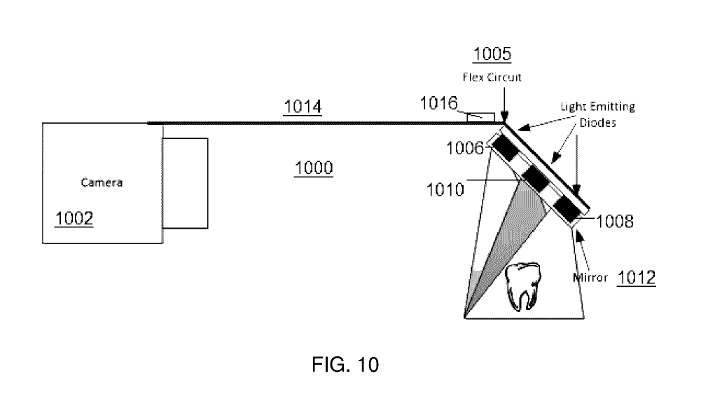

With the above as background, the subject matter of this disclosure is now described. According to this disclosure, and as shown in FIG. 10, the intra-oral device 1000 is configured as a hand-held wand that includes a monochrome 3D camera 1002, and a tip portion or “tip” 1004. As depicted, the outer housing is omitted for clarity. The tip 1004 in this embodiment houses several light emitting diodes (LEDs), such as red LED 1006, green LED 1008, and blue LED 1010. There may be multiple ones of these colored LEDs. The LEDs are mounted on a flex circuit 1005, which may include other control electronics. The tip 1004 also includes a mirror 1012, which reflects the projection path from a long axis of the device (the optical sub-system shown in FIG. 4) towards the target area being scanned, and that receives the imaging path data returned from the target area. The returned data is forwarded down the long axis of the device, where it is imaged by the CCD sensor device. In the FIG. 5 embodiment, a heating element in the tip (not shown here) received power from a conductive element 1014. According to this disclosure, signals provided over the conductive element 1014 are also used to strobe the red, green and blue LEDs, as shown. In this alternative, a separate conductor may be provided along the outer housing to power the LEDs.

In operation, the electronics (described above) synchronize the LEDs 1006, 1008 and 1010 to the frame capture of the monochrome camera 1002. The PC based software (also described above) then creates a color image by combining the red, blue, and green illuminated images. In a preferred embodiment, a microprocessor 1016 is included on the flex circuit 1005 that controls whether the LEDs are on or off. The microprocessor 1016 is connected to the conductive element bus 1014, which is normally used by the electronics to monitor the tip temperature. In operation, the device firmware is modified to send a command to the microprocessor at the beginning of every digitizing sequence. The commands may also be sent on a frame boundary. Once the microprocessor receives the command, it starts a time sequence of the LEDs. In this manner the illumination of the LEDs is synchronized to the image frames of the camera. An alternative is to place a photodetector or pin diode to monitor the illumination generated by the scanner during digitizing and derive a synchronization signal from this. The sequence generated by the microprocessor can set the delay between the LEDs turning on and the duration a specific color LED is on. By changing the duration of specific colors the white point of the resulting image can be manipulated.

In addition, the LEDs may be turned on together to increase the overall illumination. Color can be derived from Red-Green (Yellow), Blue-Red (Magenta), Green-Blue (Cyan) illumination sequence. To compensate for color shifts due to the distance the scene is from the illumination source, the 3D data be used to compensate color.

While the preferred implementation involves modifying only the scanner tip assembly (e.g., thereby enabling backward compatibility), this is not a limitation.

As variants, the LEDs may be mounted behind the mirror (using a partial reflective mirror), on the edge of the mirror, behind the mirror if a portion of the reflective coating is removed, in-between the camera and mirror, and on the camera itself. A lens may be placed in front of the LEDs to narrow the field of view (FOV) and increase illumination on the scene. Another option is to create a molded lens out of plastic, mount LEDs behind the lens, and place the entire assembly in the throat of the tip. A still further option is to place the LEDS with or without lenses in the tip mount.

A still more complex implementation uses a projector mounted alongside the camera to project colors on the scene. An advantage of this latter method is more uniform illumination of the scene. By controlling or calibrating the illumination sources, accurate color matching can also be done. A further enhancement is to place a photodetector or pin diode, or other optical sensor that observes the illumination. The sensor may be placed behind a mirror or capture stray illumination. The accuracy of the color matching is enhanced by determining the actual magnitude of the LED source. This latter approach compensates for intensity variations over temperature and age.

Accuracy may be further enhanced by measuring the current versus intensity curve of the LEDs before scanning. This allows the modulation of the LED intensity to optimize camera performance for varying scene colors and reflection constant. The exact intensity is known by setting the current of the LED. This eliminates having to dynamically measure the power of the LED during data collection.

The technique can be implemented with both far field and near field illumination.

It is not required that all three color LEDs be used, as in certain circumstances it may be sufficient just to illuminate the scene with a single color.

The subject matter herein provides numerous advantages. Generally, it provides a method for allowing existing monochrome cameras to capture color images without making hardware changes to the camera. The technique thus allows for the addition of color to products (such as the intra-oral scanner) that otherwise use monochrome imagery. As has been described, the technique creates color imagery from a monochrome camera by illuminating the scene with specific colors while the camera captures images. The color of the illumination is changed as needed. In this manner, single colored images are captured that can be combined into full color images. The monochrome camera with color tip assembly captures the color images with full resolution and sensitivity, thus producing higher quality full color images.

More generally, the display method is implemented using one or more computing-related entities (systems, machines, processes, programs, libraries, functions, code, or the like) that facilitate or provide the above-described functionality. Thus, the wand (and its system architecture) typically interface to a machine (e.g., a device or tablet) running commodity hardware, an operating system, an application runtime environment, and a set of applications or processes (e.g., linkable libraries, native code, or the like, depending on platform), that provide the functionality of a given system or subsystem. The interface may be wired, or wireless, or some combination thereof, and the display machine/device may be co-located (with the wand), or remote therefrom. The manner by which the display frames are received from the wand is not a limitation of this disclosure.

In a representative embodiment, a computing entity in which the subject matter implemented comprises hardware, suitable storage and memory for storing an operating system, one or more software applications and data, conventional input and output devices (a display, a keyboard, a gesture-based display, a point-and-click device, and the like), other devices to provide network connectivity, and the like.

Generalizing, the intra-oral digitizer wand of this disclosure is associated with the workstation to obtain optical scans from a patient's anatomy. The digitizer scans the restoration site with a scanning laser system and delivers live images to a monitor on the workstation. The techniques of this disclosure thus may be incorporated into an intra-oral digital (IOD) scanner and associated computer-aided design system, such as E4D Dentist™ system, manufactured by D4D Technologies, LLC. The E4D Dentist system is a comprehensive chair-side CAD CAM system that produces inlays, onlays, full crowns and veneers. This commercial product is also now known as Planmeca Planscan. A handheld laser scanner in the system captures a true 3-D image either intra-orally, from impressions or from models. Design software in this system is used to create a 3-D virtual model.

Generalizing, a display interface according to this disclosure is generated in software (e.g., a set of computer program instructions) executable in at least one processor. A representative implementation is computer program product comprising a tangible non-transitory medium on which given computer code is written, stored or otherwise embedded. The display interface comprises an ordered set of display tabs and associated display panels or “viewports.” Although the illustrative embodiment shows data sets displayed within multiple viewports on a single display, this is not a limitation, as the various views may be displayed using multiple windows, views, viewports, and the like. The display interface may be web-based, in which case the views of displayed as markup-language pages. The interface exposes conventional display objects such as tabbed views, pull-down menus, browse objects, and the like.

Although not meant to be limiting, the technique described above may be implemented within a chair-side dental item CAD/CAM system.

While the above describes a particular order of operations performed by certain embodiments of the described subject matter, it should be understood that such order is exemplary, as alternative embodiments may perform the operations in a different order, combine certain operations, overlap certain operations, or the like. References in the specification to a given embodiment indicate that the embodiment described may include a particular feature, structure, or characteristic, but every embodiment may not necessarily include the particular feature, structure, or characteristic. Further, while given components of the system have been described separately, one of ordinary skill will appreciate that some of the functions may be combined or shared in given systems, machines, devices, processes, instructions, program sequences, code portions, and the like.

While the techniques of this disclosure have been described in the context of a commercial intra-oral scanner such as Planmeca Planscan, this is not a limitation. Moreover, the approach may be designed and built into the monochrome camera system in the first instance as opposed to be applied as a retrofit to an existing system. Further, the technique of this disclosure may be applied with respect to any monochrome camera source.

Having described our invention, what we now claim is as follows.

1. An apparatus, comprising:
a housing supporting a monochrome camera operative to capture a scene;
a tip assembly supported in the housing, the tip assembly including a set of colored light emitting diodes (LEDs);
electronics associated with the housing to drive the light emitting diodes to illuminate the scene being captured by the monochrome camera with one or more colors; and
computer memory storing computer program instructions operative to adjust a frame capture from the monochrome camera based on illumination provided by the colored LEDs to generate a color image.

2. The apparatus as described in claim 1 wherein the set of colored LEDS comprise a red LED, a green LED and a blue LED.

3. The apparatus as described in claim 2 wherein the one or more colors are red, green and blue.

4. The apparatus as described in claim 1 wherein the one or more LEDs are strobed by control signals provided to the LEDs over a conductive element.

5. The apparatus as described in claim 4 wherein the tip assembly also includes a heating element that receives control signals over the conductive element.

6. The apparatus as described in claim 1 wherein the tip assembly also includes a mirror.

7. The apparatus as described in claim 1 wherein the mirror is partially reflective and at least one LED is mounted behind the mirror.

8. The apparatus as described in claim 1 wherein the LEDs are actuated in synchronization to the frame capture of the monochrome camera.

9. The apparatus as described in claim 1 wherein the LEDs are actuated one color at a time.

10. The apparatus as described in claim 1 wherein different color LEDs are actuated together.

11. The apparatus as described in claim 1 further including a microprocessor supported in association with the one or more LEDs to control actuation of the one or more LEDs.

12. The apparatus as described in claim 11 wherein the microprocessor delays actuating a particular LED to adjust a white point of a resulting image captured by the monochrome camera.

13. The apparatus as described in claim 11 wherein the microprocessor adjusts an intensity of an LED during image capture by the monochrome camera.

14. A system, comprising:
a monochrome camera operative to capture a scene;
a light source operative as the scene is captured by the monochrome camera to illuminate the scene with one or more colors; and
computer memory storing computer program instructions executed by a processor and operative to adjust a frame capture from the monochrome camera based on illumination provided by the light source to generate a full color image.

15. The system as described in claim 14 wherein the light source comprises a set of colored light emitting diodes (LEDs).

16. The system as described in claim 14 wherein the light source comprises a color projector.
